(19) United States
(12) Patent Application Publication
(10) Pub. No.: US 2004/0038882 A1
(43) Pub. Date: Feb. 26, 2004

(54) Sgk2 and sgk3 used as diagnostic and therapeutic targets

(76) Inventors: Florian Lang, Tuebingen (DE); Philip Cohen, Dundee (DE); Bjoern Friedrich, Tuebingen (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/362,930

(22) PCT Filed: Aug. 28, 2001
(86) PCT No.: PCT/EP01/09890

(30) Foreign Application Priority Data
Aug. 28, 2000 (DE) 100 42 137.7

(51) Int. Cl. G01N 33/53; G01N 33/567; A61K 38/16

(52) U.S. Cl. 514/12; 435/7.2

(57) ABSTRACT

The invention relates to the use of a substance, which detects sgk2 and/or sgk3, for diagnosing diseases connected with a disturbance of ion channel activity, in particular, sodium and/or potassium channels. The invention also relates to the use of an active ingredient for treating the aforementioned diseases, said active ingredient influencing the expression and/or the function of sgk2 and/or sgk3 and regulating the elimination of Na⁺ and/or K⁺ as a result.

The invention relates to use of a substance for diagnostic detection of serum-and-glucocorticoid-dependent kinase 2 and/or 3 (sgk2 and/or sgk3) and use of an active ingredient for affecting sgk2 and/or sgk3 for the therapeutic treatment of diseases correlated to perturbed ion-channel activities, in particular, those of sodium and/or potassium channels. The sgk involved represent a serine/threonine protein-kinase family that is both transcriptionally and post-transcriptionally regulated.

A variety of external signals to which cells are subject within their environments cause intracellular phosphorylation/dephosphorylation cascades in order to allow rapidly, reversibly, transmitting the signals involved from the plasma membrane and its receptors to the cytoplasm and cell nucleus. Regulation of individual proteins participating in those cascades is what initially allows the high specificities and flexibilities of the cells that allow them to rapidly respond to extracellular signals. Serum-and-glucocorticoid-dependent kinase (sgk) was originally cloned from rat mammary-carcinoma cells (Webster, et al, 1993a, 1993b). Human kinase (hsgk) was cloned in the form of a cell-volume-regulated gene from liver cells (Waldegger, et al, 1997). It was found (Chen, et al, 1999; Náray-Fejes-Toth, et al, 1999) that rat kinase stimulated the epithelial Na⁺-channel (ENaC). It was also shown (Warnock, 1998) that hypertension was accompanied by an enhanced activity of the ENaC.

hsgk is also expressed in the brain (Waldegger, et al, 1997), where the voltage-dependent K⁺-channel, Kv1.3, plays a decisive role in regulating neuronal stimulatability (Pongs, 1992). Kv1.3 also plays a major role in regulating cell proliferation (Cahalan and Chandy, 1997) and apoptotic cell death (Szabo, et al, 1996; Lang, et al, 1999). Kv1.3 is also important in the regulation of lymphocyte proliferation and function (Cahalan and Chandy, 1997). Two other members of the sgk-family, sgk2 and sgk3, were recently cloned (Kobayashi, et al, 1999). As in the case of sgk1, sgk2 and sgk3 are also activated by insulin and IGF1 via the PI3 kinase pathway. However, no other characterization or functional assignment of either of these new kinases has transpired to date.

The invention thus addresses the problem of rendering these two kinases, sgk2 and sgk3, useful for diagnostic and therapeutic purposes.

Surprisingly, in experiments employing a dual-electrode voltage clip, it could be shown that coexpression of hsgk2 or hsgk3 caused a huge increase in the activity of the epithelial Na⁺-channel (ENaC). The ENaC plays a decisive role in renal elimination of Na⁺, which, in turn, affects blood pressure. The kinase sgk3 is also expressed in the brain. In experiments employing a dual-electrode voltage clip, it could also be shown that coexpression of hsgk2 or hsgk3 caused a huge increase in the activity of the K⁺-channel, Kv1.3. Since the activation of K⁺-channels causes a reduction in neuronal excitability, the functional data that were obtained indicate that the effects of sgk3 are suitable for reducing the excitability of neurons. Perturbed expression or function of sgk3 may thus be the cause of the occurrence of epileptic attacks. Conversely, the conclusion that stimulators of the expression or activity of sgk3 that cross blood- -brain barriers may be successfully employed in the case of epileptic attacks is justified. Finally, it was found that the K⁺-channel, minK, expressed in the heart was activated by sgk1, sgk2, and sgk3. These kinases thus play a role in regulating cardiac excitability.

In view thereof, the problem addressed by the invention has been solved by the subjects of the independent claims 1, 2, 13, and 17. Preferred embodiments are stated in the dependent claims 3-12, 14-16, and 18-23. The contents of all of said claims are herewith made an integral part of the contents of this description by way of reference thereto.

According to the invention, at least one substance may be used for detecting the expression and/or function of sgk2 or sgk3 in eucaryotic cells, which will, in particular, also allow diagnosing diseases correlated to perturbed ion-channel activities, such as those of sodium and potassium channels. This substance might be, e.g., an antibody, that is directed against sgk2 or sgk3 and may be employed in a detection method, such as enzyme-linked immunosorbent assay (ELISA), that is known to specialists in the field. In the case of such immunoassays, the particular antibodies (or, in the case of antibody determinations, homologous test antigens) directed against the antigens (sgk2 and sgk3) to be detected are bound to a carrier substance (e.g., cellulose or polystyrene), on which immunocomplexes form following incubation, together with the sample. These immunocomplexes receive a marked antibody in a subsequent step. Adding a chromogenic substrate to the basic reactants allows making the enzyme-substrate complexes bound to these immunocomplexes visible or determining the antigen concentration in the sample by photometrically determining the concentrations of marker enzymes bound to these immunocomplexes through comparisons to standards having known enzymatic activities. Other substances that may be employed for diagnostic-detection purposes are the oligonucleotides, which, with the aid of polymerase chain reactions (PCR), are suitable for yielding quantitative detections of sgk2 and sgk3 using a molecular-genetical method, under which certain DNA-segments are selectively amplified. Other methods for quantitatively detecting a known target protein are well-known to specialists in the field.

In accordance with the invention, patent rights for an active ingredient for affecting, in particular, inhibiting or activating, the expression and/or function of sgk2 and sgk3 in eukaryotic cells for the purpose of treating diseases correlated to perturbed ion-channel activities, in particular, those of sodium and/or potassium channels, are claimed. Since sgk2 and sgk3 are kinases, substances, such as staurosporine, chelerythrine, etc., that are known kinase inhibitors, as well as other substances, represent candidates for that active ingredient. Such inhibitors are known to specialists in the field, and some of them are commercially available from companies, such as Sigma or Merck. Genetically altered mutants of sgk2 and/or sgk3 may, for example, be used as activators.

According to the invention, the ion channel involved may be a sodium channel of subtype ENaC, where the inhibition or activation of sgk2 and/or sgk3 preferably affects Na⁺-transport through that channel, which affects, for example, blood pressure. Hyperexpression or hyperactivity of the sgk2 and/or sgk3 causes renal retention of Na⁺, which, in turn, causes development of hypertension. Blood pressure may thus be regulated by activating or deactivating the associated kinases.

In the case of a preferred embodiment, the ion channel involved is a potassium channel of subtype Kv1.3. The effect involved, in particular, inhibition or activation of sgk2 and/or sgk3, preferably affects K⁺-transport through the potassium channel of subtype Kv1.3. In the case of other preferred embodiments, the ion channel involved is a potassium channel of subtype minK, where, in this case, inhibiting or activating sgk1, sgk2, and/or sgk3 affects K⁺-transport through the potassium channel of subtype minK.

In the case of a preferred embodiment of the invention, the active ingredient is directed against sgk2 and/or sgk3 themselves. The active ingredients involved may thus be antisense sequences, termed “kinase-deficient mutants,” or kinase inhibitors, such as staurosporine and/or chelerythrine or their analogs. So-called “small molecular compounds” or polynucleotides that encode a peptide that affects the expression of sgk2 and/or sgk3 may also be used.

In the case of another preferred embodiment of the invention, the active ingredient is directed against activators, inhibitors, regulators, and/or biological precursors of sgk2 and/or sgk3. These activators, inhibitors, regulators, and/or biological precursors might be upstream and downstream members of the sgk signal-transduction cascade, transcription factors that are responsible for sgk2-expression levels and/or sgk3-expression levels, or, as yet, unknown molecules that are affected by the active ingredient and participate in the expression and/or function of sgk2 and/or sgk/3.

The invention allows employing both known and, as yet, unknown active ingredients. In the case of a particularly preferable embodiment thereof, the active ingredient is a so-called “small molecular compound,” in particular, such having a molecular weight, MW, of MW<1,000. The “small molecular compounds” involved may also be kinase inhibitors, such as the imidazole derivatives SB 203580, which has a MW of 377.4, or SB 202190, which has a MW of 331.3, both of which are known kinase-expression inhibitors and are commercially marketed by Calbiochem, San Diego, Calif., USA.

The invention may be used for treating all forms of diseases that are correlated to perturbed sodium-channel and/or potassium-channel activities. Particularly worthy of note here are arterial hypertension, as well as symptoms corresponding to the Liddle syndrome, a rare, genetically conditioned, ENaC-hyperactivity, and thus an ailment accompanied by a huge increase in blood pressure.

So far as is presently known, diseases treatable by means of the invention that are correlated to perturbed potassium-channel activity, in particular, the activities of potassium channels of subtypes Kv1.3 and/or minK, include epilepsy, neurodegeneration, autoimmune diseases, and immunodeficiency. In particular, disorders of the minK-channel cause cardiac-rhythm fluctuations.

The invention also relates to a diagnostic kit comprising at least one substance suitable for detecting the expression and/or function of sgk2 and/or sgk3 for the purpose of diagnosing diseases correlated to perturbed ion-channel activities, in particular, those of sodium and/or potassium channels. Such a kit may also be used for diagnosing diseases correlated to hyperexpression, hypoexpression, hyperfunction, or hypofunction of sgk2 and/or sgk3. Such diagnostics may be used in conjunction with a diagnostic kit in order to detect diseases, such as arterial hypertension, Liddle syndrome, autoimmune diseases, and immunodeficiency. In this latter case as well, diseases are detected by detecting a perturbed expression and/or function of sgk2 and/or sgk3.

The invention also comprises a pharmaceutical composition containing at least one active ingredient that affects, in particular, inhibits or activates, the expression and/or function of sgk2 and/or sgk3, and, preferably, a pharmaceutical carrier, if necessary. The active ingredient involved might be a kinase inhibitor, such as the aforementioned staurosporine, chelerythrine, SB 203580, SB 202190, one of their analogs, or some other substance. The active ingredient involved might also be a polynucleotide that encodes a peptide, preferably a polypeptide, that affects, preferably inhibits or activates, the expression of sgk2 and/or sgk3. This polypeptide might, for example, be a so-called “kinase-deficient mutant.” The active ingredient involved might also be a so-called “small molecular compound,” preferably a small molecular having a molecular weight, MW, of MW<1,000. Finally, the active ingredient involved might also be an antisense sequence, i.e., a sequence that, together with mRNA, is capable of forming a double-strand duplex and thereby preventing translation of the mRNA into a polypeptide. The sequence of sgk2 and sgk3 themselves might also be used in order to yield a overexpression of these kinases by, e.g., incorporating vectors having strong promoters. Regarding the other characteristics of such a composition, reference is made to the relevant sections of the foregoing text of this description.

Finally, the invention comprises a pharmaceutical composition containing an effective quantity of at least one active ingredient that affects, in particular, inhibits or activates, the expression and/or function of activators, inhibitors, regulators, and/or biological precursors of sgk2 and/or sgk3. This pharmaceutical composition might, preferably, also contain a pharmaceutical carrier. These activators, inhibitors, regulators, and/or biological precursors of sgk2 and/or sgk3 might, e.g., be other kinases that participate in the regulation or activity of sgk2 and/or sgk3. Transcription factors that are responsible for the expression levels of sgk2 and/or sgk3, as well as other known or, as yet, unknown, members of the sgk2 and/or sgk3 signal-transduction cascade. Such compositions might also contain polynucleotides that encode a peptide that affect, in particular, inhibit or activate, the expression of activators, inhibitors, regulators, and/or biological precursors of sgk2 and/or sgk3. So-called “small molecular compounds” that preferably have molecular weights, MW, of MW<1,000 and are directed against activators, inhibitors, regulators, and/or biological precursors of sgk2 and/or sgk3, and thereby inhibit or activate the expression or function of those kinases may also be employed.

The existing features and other features of the invention arise from the following descriptions of preferred embodiments thereof, together with the subclaims and figures, where the individual features thereof may be implemented either alone, or in combinations with each other.

The figures depict:

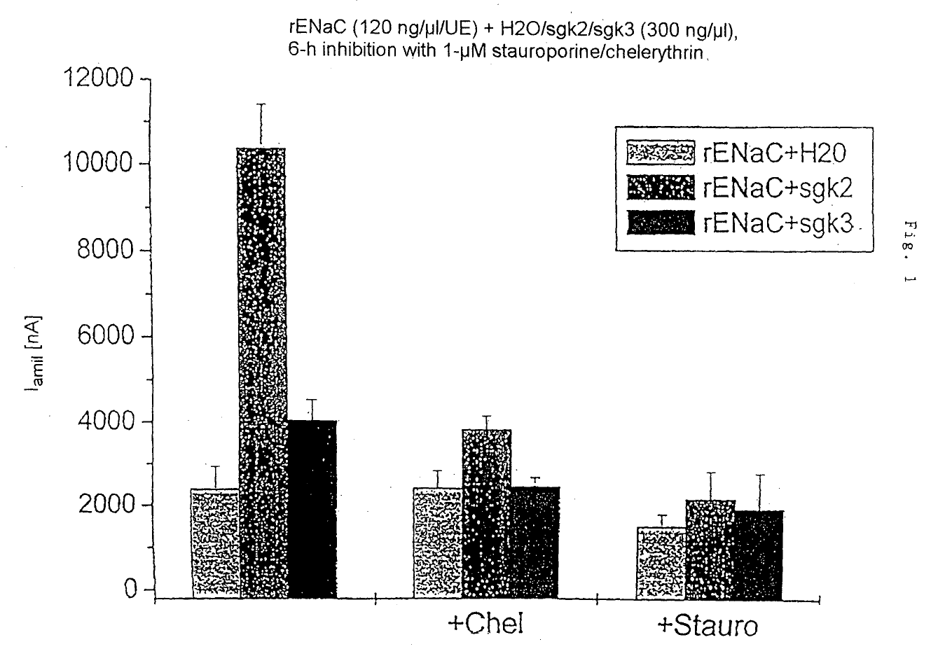

FIG. 1: Stimulation of the Na⁺-channel, rENaC, by hsgk2 and hsgk3.

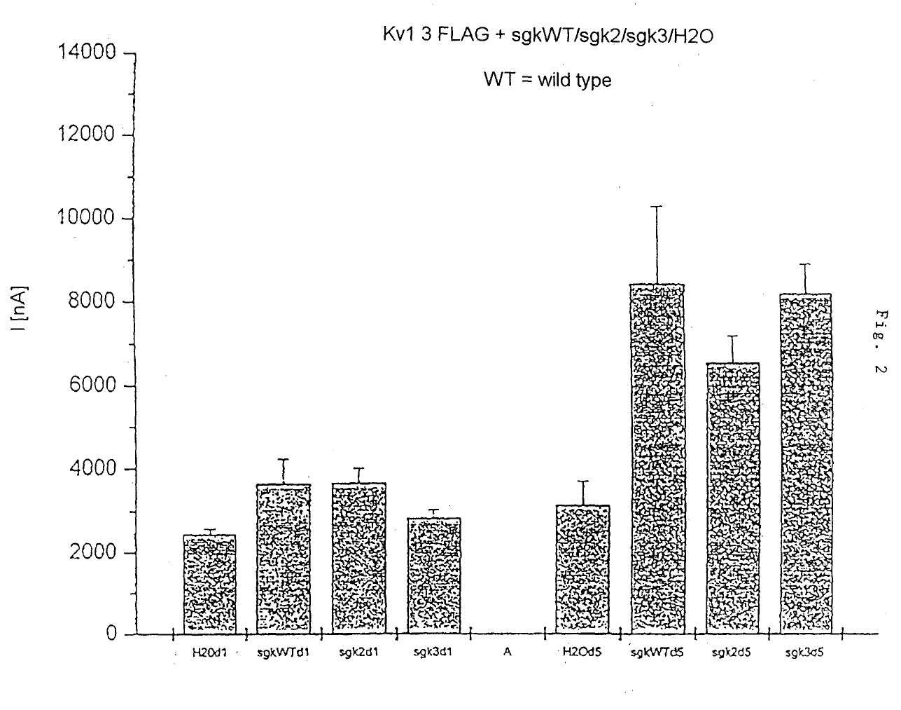

FIG. 2: Stimulation of the K⁺-channel, Kv1.3, by hsgk2 and hsgk3.

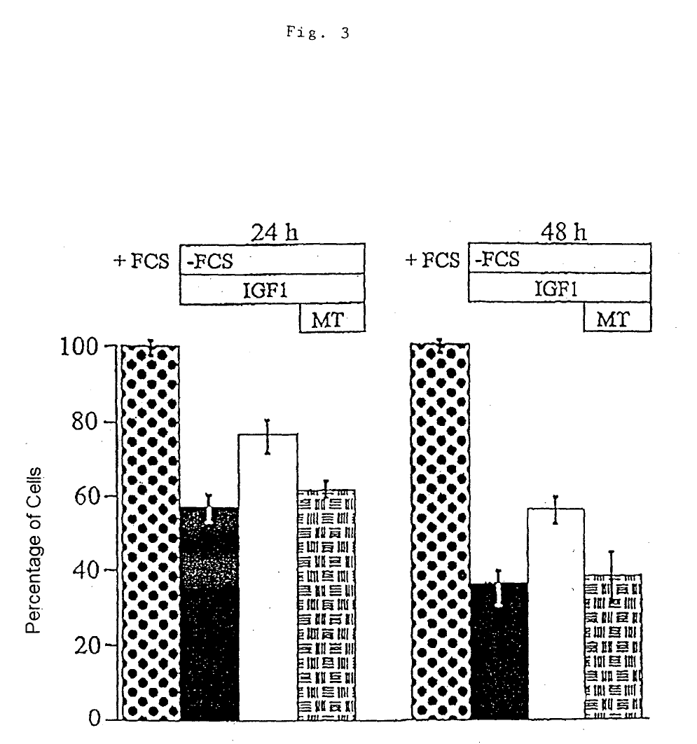

FIG. 3: The effect of inhibition of the K⁺-channel, Kv1.3, on the survival of human embryonic kidney cells (HEK-cells).

### MATERIALS AND METHODS

The dissection of Xenopus laevis and the recovery and treatment of oocytes have been described in detail earlier (Busch, et al, 1992). Each of the oocytes involved was injected with 1 ng cRNA from α-ENaC, β-ENaC, and γ-ENaC, Kv1.3, or minK, both with and without simultaneous injection of the kinases hsgk1, hsgk2, and hsgk3. Dual-electrode voltage-clip and current-clip experiments could be undertaken 2 to 4 days following injection. Na⁺-currents (in the case of ENaC) and K⁺-currents (in the case of Kv1.3 and minK) were filtered at 10 Hz and recorded using a recorder. The experiments were normally conducted on the second day following cRNA-injection. The bath solution contained 96 mM NaCl, 2 mM KCl, 1.8 mM CaCl₂, 1 mM MgCl₂, and 5 mM HEPES at a pH of 7.5 and a holding potential of −80 mV. In all experiments, bath pH was set by titration with HCl or NaOH. Bath-liquid flow rate was set to 20 ml/min, which provided for a complete change of solution within 10 to 15 seconds. All data were output in the form of arithmetic means±SEM.

### RESULTS

In order to investigate the effects of hsgk1, hsgk2, or hsgk3, the mRNA of the respective kinases, together with the mRNA of the epithelial Na⁺-channel, α-ENaC, β-ENaC, and γ-ENaC, or the voltage-dependent K⁺-channel, Kv1.3, or the minK-channel, were injected into Xenopus oocytes and the amiloride-sensitive Na⁺-current, I_{Na}, and voltage-activated K⁺-current, I_{K}, subsequently determined. As may be seen from Table 1, below, and FIGS. 1 and 2, both hsgk2 and hsgk3 stimulate ENaC-activity and Kv1.3-activity. hsgk1 stimulates minK-activity (cf. Table 1). Their stimulating effects were totally prevented by the protein-kinase inhibitors staurosporine and chelerythrine.

TABLE 1

| | No Kinase | hsgk1 | hsgk2 | hsgk3 | n |
|---|---|---|---|---|---|
| α-ENaC, | 2.5 ± 0.3 | 5.9 ± 1.0 | 9.4 ± 1.7 | 4.5 ± 0.8 | 7 |
| β-ENaC, | | | | | |
| and γ- | | | | | |
| ENaC I_{Na} | | | | | |
| Kv1.3 I_{K} | 3.1 ± 0.6 | 8.4 ± 1.8 | 6.5 ± 0.6 | 8.2 ± 0.7 | 7 |
| MinK I_{K} | 0.67 ± 0.07 | 1.16 ± 0.11 | 0.97 ± 0.1 | 1.1 ± 0.11 | 7 |

Table 1:
Na⁺-currents (I_{Na}) [μA] and K⁺-currents (I_{K}) [μA] in oocytes that have been injected with (deionized) water, with α-ENaC, β-ENaC, and γ-ENaC, with Kvl.3, or with minK, containing, or not containing, hsgk1, hsgk2, or hsgk3.

### EXPERIMENT 1

Following injection of the mRNA from hsgk2 and hsgk3, it could be shown (cf. FIG. 1) that the amiloride-inhibitable current, Iₐₘᵢₗ, through the Na⁺-channel, rENaC, increased significantly due to the coexpression with hsgk2 and hsgk3. The kinase inhibitors staurosporine and chelerythrine inhibit activation of the Na⁺-channel (cf. FIG. 1). Since the stimulating effects of the hsgk2 and hsgk3 on the ENaC-channel may be prevented by the kinase inhibitors staurosporine and chelerythrine, (a) diagnostic detection of a perturbed expression or function of sgk2 or sgk3 represent a major measure in discovering the cause of, for example, incidence of hypertension, and (b) sgk2-inhibitors and sgk3-inhibitors, such as staurosporine, chelerythrine, or other kinase inhibitors, may be employed in the therapy of the aforementioned disease.

### EXPERIMENT 2

Following injection of the mRNA from hsgk1, hsgk2, or hsgk3, together with the mRNA from the K⁺-channels Kv1.3 or minK, it could be shown that the current through either of these channels, I, may be increased (cf. Table 1). FIG. 2 presents the results of those experiments, following injection of the mRNA from hsgk2 and hsgk3, together with the mRNA from Kv1.3, obtained on the first day (d1, the leftmost bars) and fifth day (d5, the rightmost bars). Since activation of K⁺-channels causes a reduction in neuronal excitability, these functional data indicate that the effects of hsgk3 expressed in the brain are suitable for reducing the excitabilities of neurons. A perturbed expression or function of sgk3 may thus be the cause of occurrences of epileptic attacks. Conversely, stimulators of the expression or function of sgk3 that cross blood-brain barriers may be employed in the event of epileptic attacks. These same considerations apply to stimulation or inhibition of kinases, in particular, hsgk1, for the purpose of affecting perturbed cardiac excitability.

### EXPERIMENT 3

According to FIG. 3, extracting fetal calf serum (FCS) from human embryonic kidney cells (HEK-cells) (Lewis, et al (1984); Phillips, et al (1982)) reduces the total number of cells present due to cell mortality, as may be seen by comparing the black bars to the dotted bars, where FIG. 3 depicts the situations after 24 hours and 48 hours, respectively. This reduction is lessened by the insulin-like growth factor (IGF1), represented by the white bars. The effect of IGF1 is eliminated by the simultaneous inhibition of K⁺-channels using margatoxin (MT), represented by the hatched bars. These data indicate that the insulin-like growth factor, IGF1, loses its cell-death-inhibiting effect when K⁺-channels are simultaneously inhibited. The activation of the Kv1.3-channel mediated by sgk2 and sgk3 thus has an antiapoptotic effect, and lack of an sgk2-effect and sgk3-effect would thus cause an increase in cell mortality, as occurs in the case of, for example, neurodegeneration. Conversely, activators of sgk2 and sgk3 may be employed for preventing apoptotic cell death in the case of neurodegeneration. Since Kv1.3 also plays a major role in regulating lymphocyte proliferation and lymphocyte function, inhibitors or activators of these kinases may be employed for affecting the immune system in the case of, e.g., autoimmune diseases or immune deficiency.

### Literature References

A. E. Busch, M. P. Kavenaugh, M. D. Varnum, J. P. Adelman, and R. A. North: “Regulation by second messengers of the slowly activating voltage-dependent potassium current expressed in Xenopus oocytes.” J. Physiol. Lond. 450 (1992), pp. 491-502.

M. D. Cahalan and K. G. Chandy: “Ion channels in the immune system as targets for immunosuppression” Cur. Opin. Biotech. 8 (6) (1997), pp. 749-756.

S. Y. Chen, A. Bhargava, L. Mastroberardino, O. C. Meijer, J. Wang, P. Buse, G. L. Firestone, F. Verrey, and D. Pearce: “Epithelial sodium channel regulated by aldosterone-induced protein sgk.” Proc. Nat. Acad. Sci. USA 96 (1999), pp. 2514-2519.

T. Kobayashi, M. Deak, N. Morrice, and P. Cohen: “Characterization of the structure and regulation of two novel isoforms of serum-and-glucocorticoid-induced protein kinase” Biochem. J. 344 (1999), pp. 189-197.

F. Lang, I. Szabo, A. Lepple-Wienhues, D. Siemen, and E. Gulbins: “Physiology of receptor mediated lymphocyte apoptosis.” News Physiol. Sci. 14 (1999), pp.194-200.

M. L. Lewis, D. R. Morrison, B. J. Mieszkuc, and D. L. Fessler: “Problems n the bioassay of products from cultured HEK cells: plasminogen activator.” Adv. Exp. Med. Biol. 172 (1984), pp. 241-267.

A. Náray-Fejes-Toth, C. Canessa, E. S. Cleaveland, G. Aldrich, and G. Fejes-Toth: “Sgk is an aldosterone-induced kinase in the renal collecting duct. Effects on epithelial Na⁺ channels.” J. Biol. Chem. 274 (1999), pp.16973-16978.

S. G. Phillips,. S. L. Lui, and D. M. Phillips: “Binding of epithelial cells to lectin-coated surfaces.” In Vitro 18 (1982), pp. 727-738.

O. Pongs: “Molecular biology of voltage-dependent potassium channels” Physiol. Rev. 72 (1992), pp. S69-S88.

I. Szabo, E. Gulbins, H. Apfel, X. Zhan, P. Barth, A. E. Busch, K. Schlottmann, O. Pongs, and F. Lang: “Tyrosine phosphorylation-dependent suppression of a voltage-gated K⁺-channel in T lymphocytes upon Fas stimulation.” J. Biol. Chem. 271 (1996), pp. 20465-20469.

S. Waldegger, P. Barth, G. Raber, and F. Lang: “Cloning and characterization of a putative human serine/threonine protein kinase transcriptionally modified during anisotonic and isotonic alterations of cell volume.” Prof. Nat. Acad. Sci. USA 94 (1997), pp 4440-4445.

D. G. Warnock: “Liddle syndrome: An autosomal dominant form of human hypertension.” Kidney Ind. 53 (1998), pp.18-24.

M. K. Webster, L. Goya, and G. L. Firestone: “Immediate-early transcriptional regulation and rapid mRNA turnover of a putative serine/threonine protein kinase.” J. Biol. Chem. 268 (16) (1993a), pp. 11482-11485.

M. K. Webster, L. Goya, Y. Ge, A. C. Maiyar, and G. L. Firestone: “Characterization of sgk, a novel member of the serine/threonine protein kinase gene family which is transcriptionally induced by glucocorticoids and serum.” Mol. Cell Biol. 13 (4) (1993b), pp. 2031-2040.

1. Use of a substance for detecting the expression and/or function of sgk2 and/or sgk3 in eukaryotic cells for the purpose of diagnosing diseases correlated to perturbed ion-channel activities, in particular, those of sodium and/or potassium channels.

2. Use of an active ingredient for affecting, in particular, inhibiting or activating, the expression and/or function of sgk2 and/or sgk3 in eukaryotic cells for the purpose of treating diseases correlated to perturbed ion-channel activities, in particular, those of sodium and/or potassium channels.

3. Use according to claim 2, wherein the effect, in particular, inhibition or activation of sgk2 and/or sgk3, affects and/or controls the elimination of Na⁺ and/or K⁺.

4. Use according to any of the foregoing claims, wherein the ion channel is a sodium channel of subtype ENaC.

5. Use according to any of the foregoing claims, wherein the ion channel is a potassium channel of subtype Kv1.3.

6. Use according to any of claims 2-5, wherein the active ingredient is directed against sgk2 and/or sgk3.

7. Use according to any of claims 2-6, wherein the active ingredient is directed against activators, inhibitors, regulators, and/or biological precursors of sgk2 and/or sgk3.

8. Use according to any of claims 2-7, wherein the active ingredient is a kinase inhibitor, preferably staurosporine and/or chelerythrine or their analogs.

9. Use according to any of claims 2-8, wherein the active ingredient is a polynucleotide that encodes a peptide, preferably a polypeptide, where that peptide affects, preferably inhibits or activates, the expression of sgk2 and/or sgk3.

10. Use according to any of claims 2-9, wherein the active ingredient is a “small molecular compound,” preferably a “small molecular compound” having a molecular weight, MW, of MW<1,000.

11. Use according to any of the foregoing claims, wherein the diseases, in particular, diseases correlated to perturbed sodium-channel activities, are arterial hypertension or symptoms corresponding to the Liddle syndrome.

12. Use according to any of the foregoing claims, wherein the diseases, in particular, diseases correlated to perturbed potassium-channel activities, are epilepsy, neurodegeneration, auto-immune diseases, or immunodeficiency.

13. A diagnostic kit comprising at least one substance for detecting the expression and/or function of sgk2 and/or sgk3 for the purpose of diagnosing diseases correlated to perturbed ion-channel activities, in particular, those of sodium and/or potassium channels.

14. A diagnostic kit according to claim 13 for diagnosing diseases correlated to hyperexpression and/or hypoexpression of sgk2 and/or sgk3.

15. A diagnostic kit according to claim 13 or claim 14 for diagnosing arterial hypertension or symptoms corresponding to the Liddle syndrome.

16. A diagnostic kit according to claim 13 or claim 14 for diagnosing epilepsy, neurodegeneration, autoimmune diseases, or immunodeficiency.

17. A pharmaceutical composition comprising an effective quantity of at least one active ingredient that affects, in particular, inhibits or activates, the expression and/or function of sgk2 and/or sgk3 and, if necessary, a pharmaceutical carrier.

18. A pharmaceutical composition according to claim 17, wherein the active ingredient is a kinase inhibitor, preferably staurosporine and/or chelerythrine or their analogs.

19. A pharmaceutical composition according to claim 17, wherein the active ingredient is a polynucleotide that encodes a peptide, preferably a polypeptide, where that peptide affects, preferably inhibits or activates, the expression of sgk2 and/or sgk3.

20. A pharmaceutical composition according to claim 17, wherein the active ingredient is a “small molecular compound,” preferably a “small molecular compound” having a molecular weight, MW, of MW<1,000.

21. A pharmaceutical composition according to claim 17 comprising an effective quantity of at least one active ingredient that affects, in particular, inhibits or activates, the expression and/or function of activators, inhibitors, regulators, and/or biological precursors of sgk2 and/or sgk3 and, if necessary, a pharmaceutical carrier.

22. A pharmaceutical composition according to claim 21, wherein the active ingredient is a polynucleotide that encodes a peptide, preferably a polypeptide, where that peptide affects, preferably inhibits or activates, the expression of activators, inhibitors, regulators, and/or biological precursors of sgk2 and/or sgk3.

23. A pharmaceutical composition according to claim 21, wherein the active ingredient is a “small molecular compound,” preferably a “small molecular compound” having a molecular weight, MW, of MW<1,000.
